(19) United States
(12) Patent Application Publication
(10) Pub. No.: US 2017/0128613 A1
(43) Pub. Date: May 11, 2017

(54) METHOD AND SYSTEM FOR CLEANING AIR

(71) Applicant: Michael Stewart, Detroit, MI (US)

(72) Inventor: Michael Stewart, Detroit, MI (US)

(21) Appl. No.: 15/267,795

(22) Filed: Sep. 16, 2016

Related U.S. Application Data
(60) Provisional application No. 62/219,258, filed on Sep. 16, 2015.

(51) Int. Cl. A61L 9/20; A61L 9/12

(52) U.S. Cl. CPC A61L 9/20; A61L 9/12; A61L 2209/14; A61L 2209/111

(57) ABSTRACT

An apparatus for disinfecting forced air systems, having a main member including an inner surface configured for receiving the forced air systems. The main member includes a plurality of apertures disposed along the inner surface. The apparatus has at least one temperature controller and at least one air source to the controller for heated blowing air. The apertures are in communication with at least the air source, and UV light from the light source and air from the air source can at least partially pass through the inner surface.

### CROSS-REFERENCE TO RELATED APPLICATIONS

This application claims the benefit of U.S. Provisional Application No. 62/219,258, filed on Sep. 16, 2015. The entire disclosure of the above application is incorporated herein by reference

### FIELD

The present disclosure relates to a system and method for eliminating filtering air and, more particularly, to a system and method for disinfecting air traveling through a forced air system.

### BACKGROUND

The background description provided here is for the purpose of generally presenting the context of the disclosure. Work of the presently named inventor, to the extent it is described bed in this background section, as well as aspects of the description that may not otherwise qualify as prior art at the time of filing, are neither expressly nor impliedly admitted as prior art against the present disclosure.

Microbes often reside in forced air systems associated with homes and commercial buildings. While not easily detected, microbes can live on floating particulate matter such as dust.

### SUMMARY

This section provides a general summary of the disclosure, and is not a comprehensive disclosure of its full scope or all of its features.

According to the present teachings, an apparatus for disinfecting forced air systems is presented. The apparatus which defines a chamber having an inner surface configured to be positioned within a portion of forced air systems is provided. The main member defines an input aperture an output aperture fluidly coupled to a furnace. A UV-C light source and a filter are coupled to the interior surface of the chamber to apply UV-C radiation to the exterior surface of the forced air systems.

Further, according to the present teachings, an apparatus for disinfecting air within a forced air systems is provided. The apparatus has a main member having an inner surface defining a chamber. The apparatus is coupled to air direction system such as an adjustable grate. The inner surface defines a plurality of apertures fluidly coupled to a heat or cold-generating source.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The drawings described be herein are for illustrative purposes only of selected embodiments and not all possible implementations, and are not intended to limit the scope of the present disclosure.

FIG. 1 represents a perspective view of a disinfecting system according to the present teachings;

FIGS. 2A and 2B represent a disinfecting system usable in furnace filter construction;

FIGS. 3A-3D represent translucent frames usable in the systems shown in FIGS. 1-2B;

FIGS. 4A-4C represent alternate translucent frames usable in the systems shown in FIGS. 1-2B;

FIGS. 5A-5C represent alternate translucent frames usable in the systems shown in FIGS. 1-2B;

FIGS. 6A-6D represent alternate translucent frames usable in the systems shown in FIGS. 1-2B;

FIGS. 7A-7C represent sectional views of an alternate translucent frames usable in the systems shown in FIGS. 1-2B; and

FIG. 8 represents an alternate translucent frame usable in the systems shown in FIGS. 1-2B having an incorporated UV light source.

Corresponding reference numerals indicate corresponding parts throughout the several views of the drawings.

### DETAILED DESCRIPTION

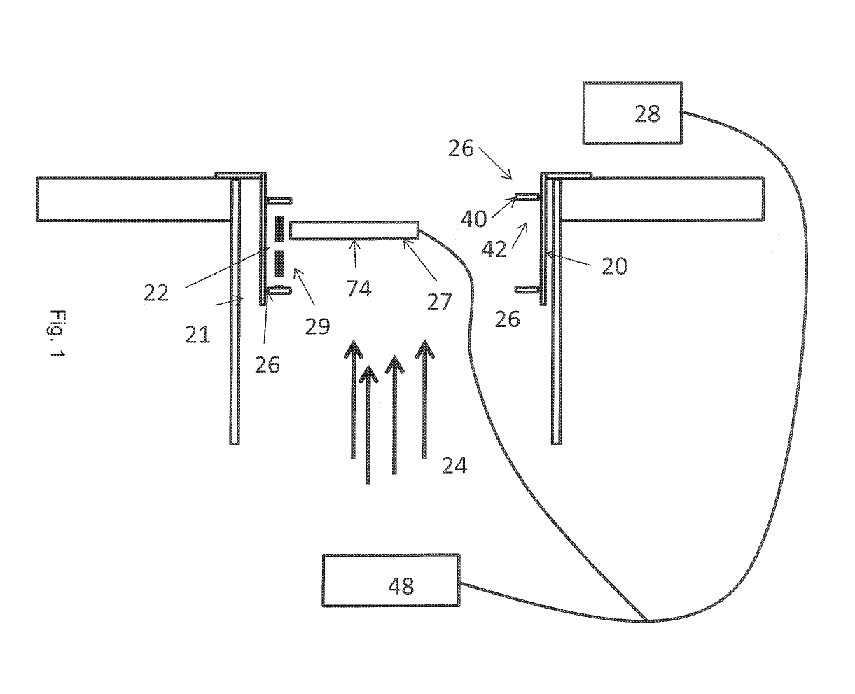

Example embodiments will now be described bed more fully with reference to the accompanying drawings. FIG. 1 represents a perspective sectional view of the disinfecting system mounted on an internal building surface according to the present teachings. The apparatus for disinfecting forced air systems, has an openable enclosure or main member 20 including an inner surface 22 configured for receiving the forced air 24. The main member 20 includes a plurality of flanges 26 disposed along the inner surface 22. At least one temperature controller 28 or thermostat is provided for controlling 28 the temperature within a chamber 30 defined by the inner surface. The controller 28 is coupled to a furnace 32 supplying heated blow in air to the chamber 30. The flanges 26 are used to support filters which are in communication with the furnace 32 or air source. Optionally, UV light from a light source and air from the furnace air source can at least partially pass through the inner surface.

According to various exemplary illustrations described bed herein, the main member 20 is provided and includes an inner surface 22. The openable enclosure provided by the main member 20 defines the cavity configured to accept a number of forced air systems Air flow which may be infected with pests such as microbes. The openable enclosure can be associated with a ceiling or wall mounted air direction vent 23, 24. The openable enclosure 20 is formed of a plurality of optionally insulated walls 21 that are configured to efficiently allow maintain an elevated airflow through the enclosure. A see better below, the main member 20 can include at least one horizontally oriented channel 29 for creating an air-low space between the main member and the forced air systems, and to facilitate the coupling of filter racks therein.

It is envisioned that the enclosure can use several different mechanisms to effect disinfection, each having its own advantage or disadvantage. These include maintaining a plurality of apertures disposed along the inner surface. At least one light source 27 for emitting a UV and preferably UV-C light, and at least one air source for blowing air are also provided. The apertures located along the inner surface of the main member are in communication with at least the air source. UV-C light from the light source (such as an OLED, SLD) and air from the air source can at least partially pass though the inner surface. The main member is constructed from a material with a transparency that allows for UV-C light from the air source to at least partially pass through. Alternatively, the light source is at least one optical fiber, wherein at least one aperture of the main member receives an end of the optical fiber, where the end emits the UV-C light. Finally, the disinfector may also include a spray source for spraying a solution, where the spray source is in communication with the apertures of the main member.

Ultraviolet irradiation in the C bandwidth (UV-C) is used for disinfection purposes because the UV-C light kills microorganisms, mold and bacteria that are trapped outside the forced air systems. In one example, the ultraviolet light source 74 is a mercury-vapor lamp that emits UV-C light; however, it should be noted that any light source that emits UV-C rays may be used as well. In one illustration, the ultraviolet light source 74 also produces ozone (O₃); however, an ultraviolet light source 74 that does not produce ozone may be used as well. Ozone and UV-C light are combined with moisture that is trapped outside the forced air systems to remove odors. More specifically, the combination of ozone and UV-C light with moisture produces chemicals, such as hydroxyl radicals (—OH), which are purifying agents that neutralize unpleasant odors that are trapped outside the forced air systems. The ultraviolet light source 74 can be powers using a power cord or a battery with power supplies.

In addition to modifying the temperature within the chamber 30, it is envisioned that other atmospheric modification can take place. The apparatus described above can have spray sources for spraying a pesticide or disinfecting solution. The spray source in communication with the apertures defined by the chamber 30. Nozzles (not shown) associated with the pesticide can fumigate the chamber, or can spray solution directly onto the forced air systems. Alternatively, nitrogen or ozone can be incorporated into the chamber to increase the effectiveness of heat in eliminating the pests.

The main member 20 may define a plurality of filter holding frames 40 which can be slid into the openable chamber for disinfection. The frames 40 can have removable flanges and can have a mechanism such as a tongue and groove coupling which will allow for the frames to be slid into channels 42 formed in the floor of the chamber 30. The channels can create an air-flow space between the main member and the forced air systems. Additionally, the channels have a coupling mechanism that allows selective engagement of the rack to the interior surface. Alternatively, the frame 40 can have a soft cover to prevent the spread of infection.

As can be seen the carrier 40 can be collapsible to assist in moving the carrier into and out of chamber. In this regard, the collapsible carrier can have soft sides and can have an accordion structure. Additionally, the frames 40 can have pin-in-slot structures to facilitate collapse of the frames 40 to accommodate filters of various sizes.

The air source 48 includes a furnace or furnace 32 or air conditioner for heating air emitted from the air source to maintain a temperature in the chamber 30. The furnace 32 is controlled by the thermostat or controller 28 to maintain the temperature within the chamber to a proper elevation. Furnace 32 can be a separate unit or can be integrated into or directly attached to the main member.

The main member 20 as shown can have a plurality of support flanges. In this regard, the main member may be a small chamber 30 slid into a vent such as a commercial building vent or a house for disinfecting the forced air systems. Additionally, as described bed above, the main member 20 can be attached to a wall or floor. The main member 20 can be substantially shaped to receive a HEPA filter or a plurality of filters therein. Alternatively, the chamber can have a plurality of disinfecting lamps which can be configured to move to project a random light onto the surface of the forced air systems. Optionally, the internal surfaces can be coated with TiO₂ which will emit ozone when subjected to UV light.

The frame member 40 can be at least partially formed made of a polymer material which is optically transparent to UV frequencies configured to transmit light from the light source 27 for emitting a UV. The apertures located along the inner surface of the main member are in communication with at least the air source. The shape of the polymer light is configured to distribute the light in an even distributed manner. Preferably, the light is configured to impinge all surfaces of dust particles with sterilizing UV light. The material can be for example a UV transmitting acrylic sheet or bar. The materials should provide high levels of UV light transmission in the UV-A (315-400 nm), UV-B (280-315 nm) and UVC (290 nm-100 nm) regions while providing strong resistance to degradation caused by UV light. Optionally, the chamber can incorporate separate the heating components 62 can be for instance a plurality of electric coils or wires whose temperature is controlled using a controller 28 or thermostat. The chamber can incorporate baffles for forced air or ozone a light pipes to transport disinfecting UV light. Optionally, a pressure or light sensor can be used to indicate the filter is clogged. Optionally, an internet connection can be provided to present feedback to a central controller to indicate the filter is plugged.

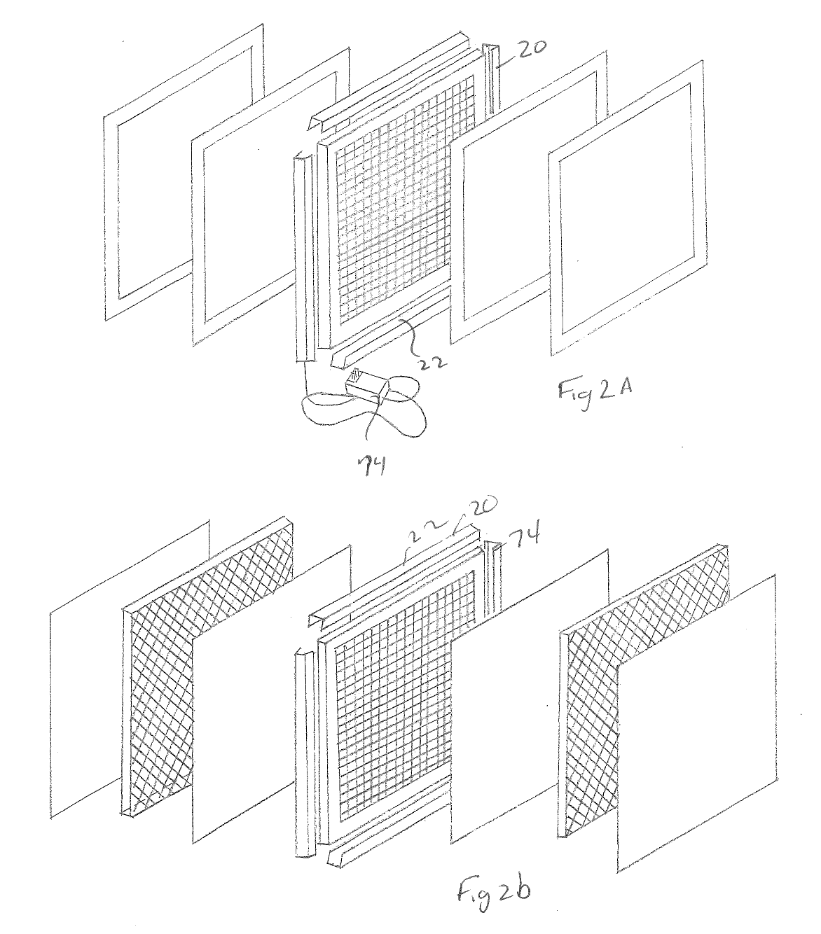

FIGS. 2A and 2B represent a disinfecting system usable in furnace filter construction. As shown, the filter frame 20 can have a transparent material and frame coupled about a filter. The ultraviolet light source 74 can be directly coupled to the frame 20 which functions as a light pipe to transfer light onto the into the air flow to disinfect particles or to a TiO2 catalyst which convers oxygen into ozone.

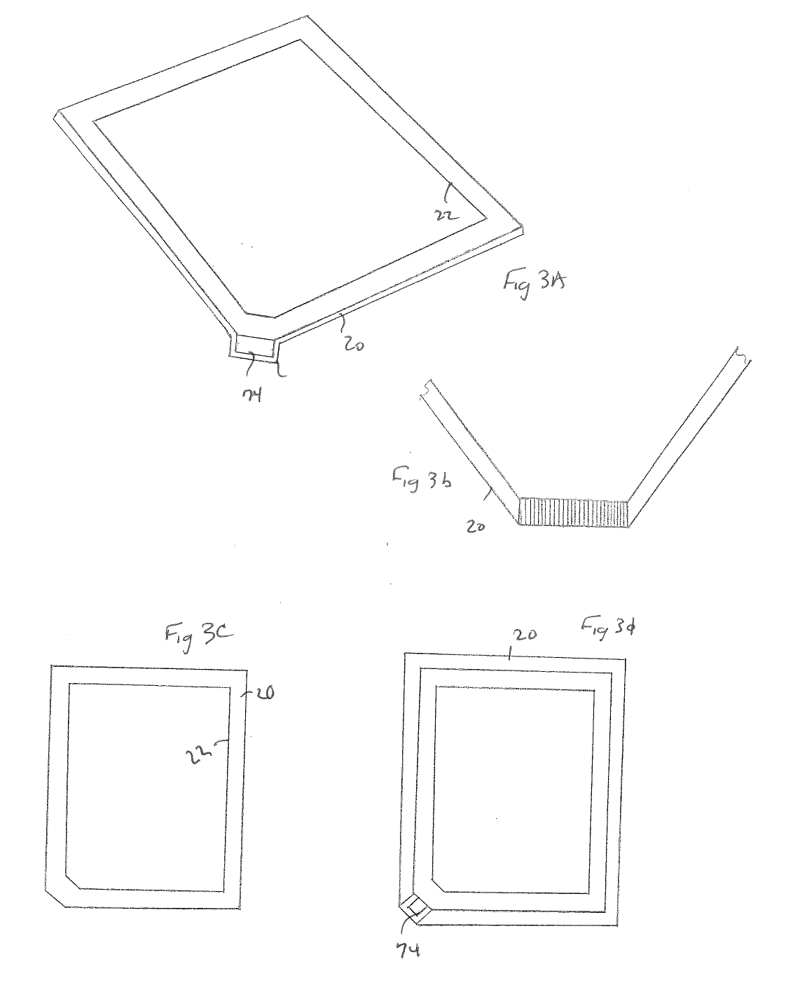

FIGS. 3A-3D represent translucent frames usable in the systems shown in FIGS. 1-2B. As shown, the filter frame 20 can have a transparent material and frame coupled about the UV light source. The ultraviolet light source 74 can be directly coupled to the frame 20 which functions as a light pipe to transfer light onto the into the air flow to disinfect particles or to a TiO2 catalyst which convers oxygen into ozone.

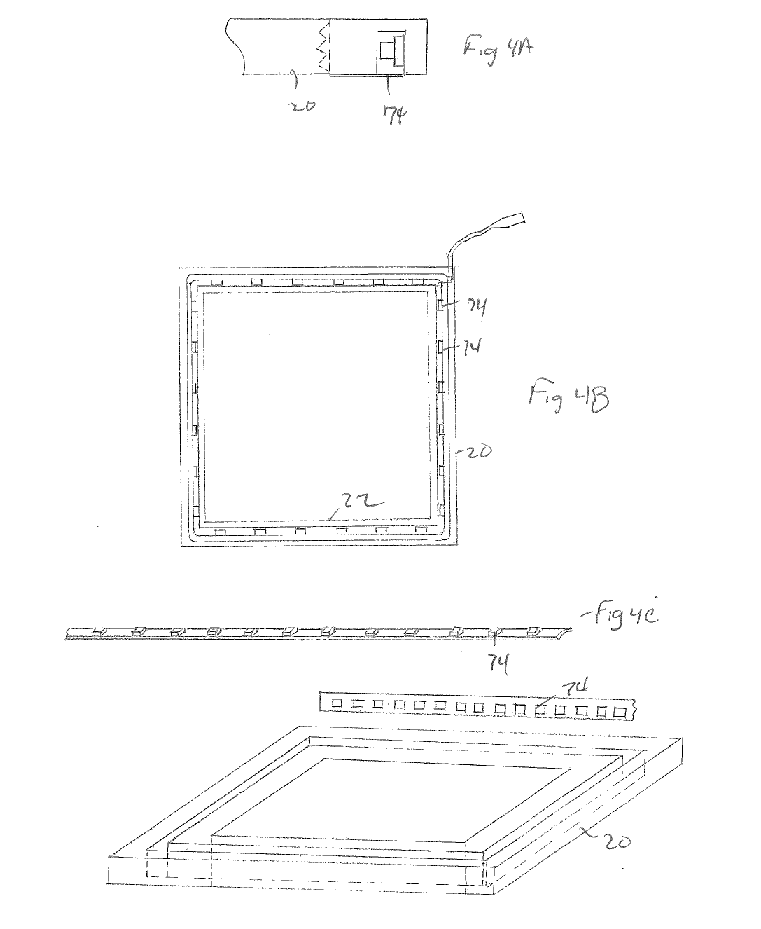

FIGS. 4A-4C represent alternate translucent frames usable in the systems shown in FIGS. 1-2B. As shown, the filter frame 20 can have a transparent material and frame coupled about a filter. The ultraviolet light source 74 or laser can be directly coupled to the frame 20 which functions as a light pipe. A plurality of LED's can be distributed in a linear array to the translucent frame.

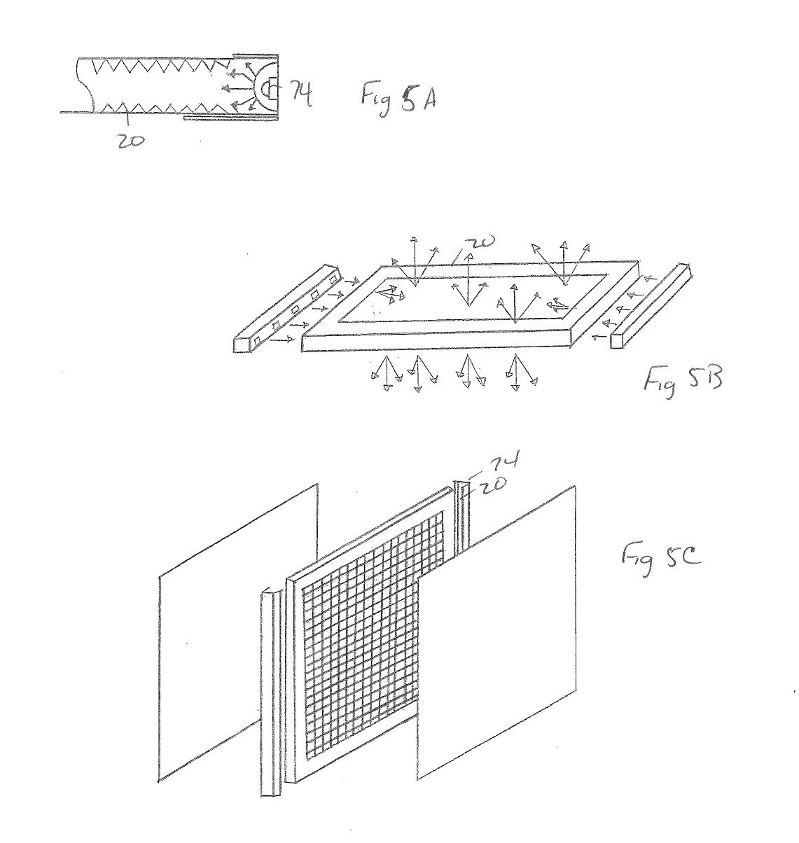

FIGS. 5A-5C represent alternate translucent emission of UV light from the frames usable in the systems shown in FIGS. 1-2B. As can be seen, the internal and external surfaces of the polymer frame which functions to disperse the light.

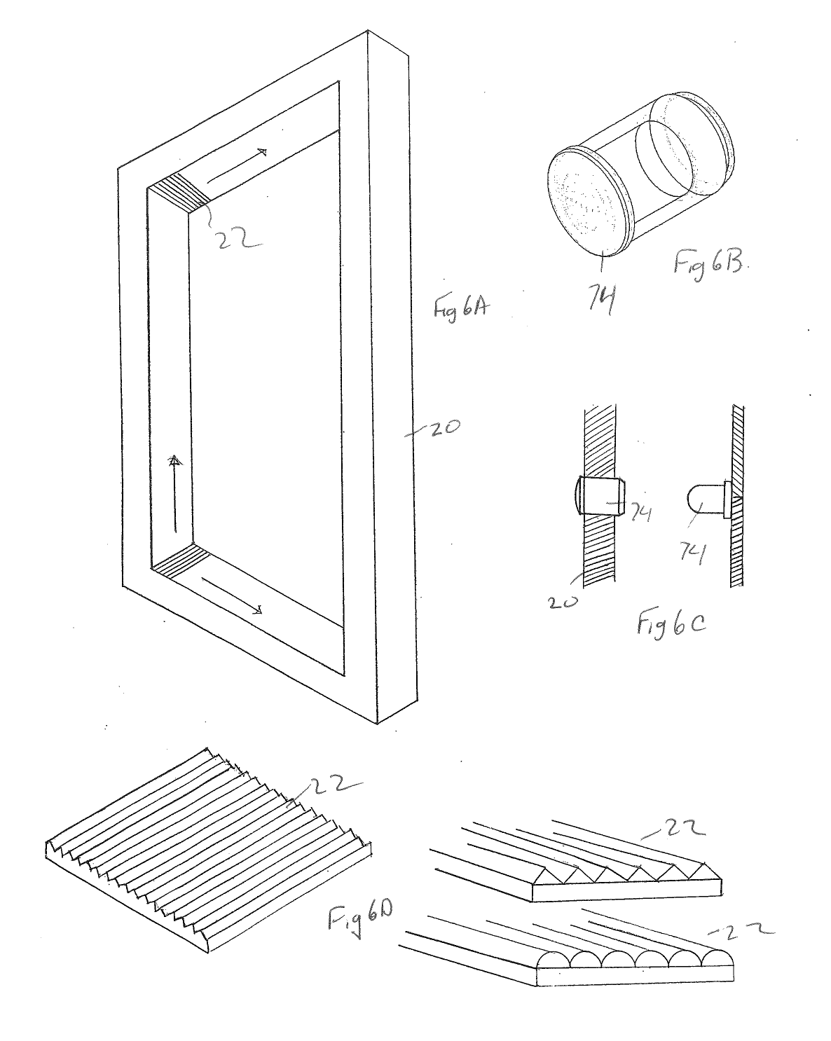

FIGS. 6A-6D represent alternate translucent frames having light dispersing surfaces usable in the systems shown in FIGS. 1-2B. As shown, the internal (or external) surfaces can have a serrated or circular surface. The light source can be disposed within the frame, or can be adjacent to the frame to inject light into the light pipe frame.

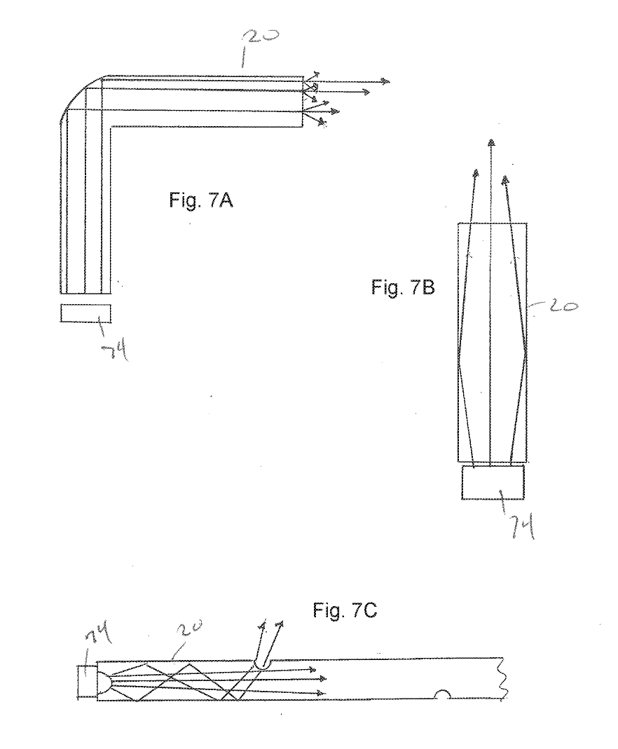

FIGS. 7A-7C represent sectional views of alternate translucent frames usable in the systems shown in FIGS. 1-2B. Shown is the frame functioning to refract light within the frame to move the light into a different position which allows dispersion into the flowing air path. The inner surface 22 can have a convex or concave lens to transmit the light into a plane or a cone.

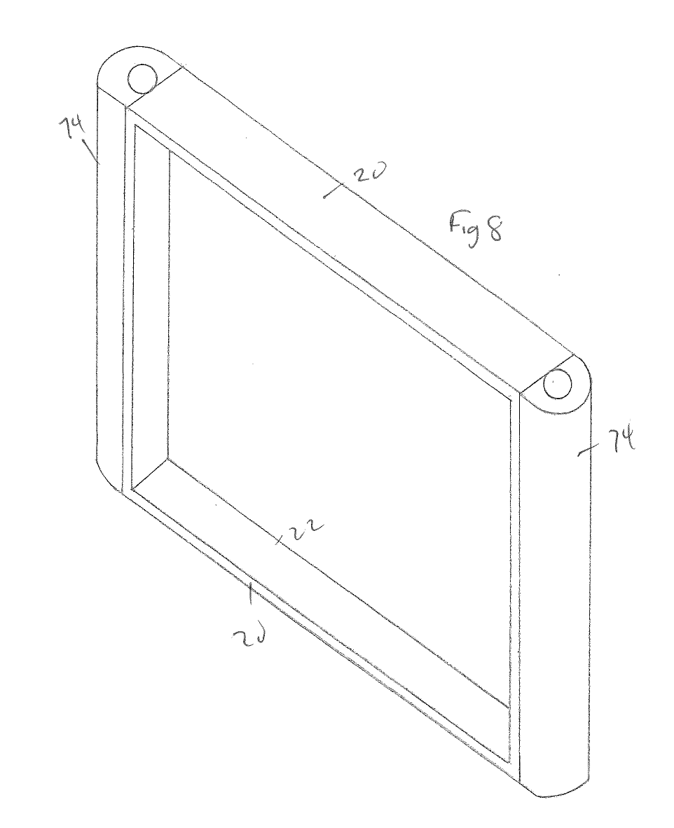

FIG. 8 represents an alternate translucent frame usable in the systems shown in FIGS. 1-2B having an incorporated UV light source. Shown are light sources 74 which can have a plurality of LED sources coupled to the frame.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of described bring particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to described be various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to described be one element or feature's relationship to another element(s) or feature(s) as illustrated in the figure. Spatially relative terms may be intended to encompass different orientations of the device in use or operation in addition to the orientation depicted in the figure. For example, if the device in the figure is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative description used herein interpreted accordingly.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. As used herein, the phrase at least one of A, B, and C should be construed to mean a logical (A or B or C), using a non-exclusive logical OR. It should be understood that one or more steps within a method may be executed in different order (or concurrently) without altering the principles of the present disclosure.

In this application, including the definitions below, the term controller may be replaced with the term circuit. The term module may refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, or group) that executes code; memory (shared, dedicated, or group) that stores code executed by a processor; other suitable hardware components that provide the described functionality; or a combination of some or all of the above, such as in a system-on-chip.

The term code, as used above, may include software, firmware, and/or microcode, and may refer to programs, routines, functions, classes, and/or objects. The term shared processor encompasses a single processor that executes some or all code from multiple modules. The term group processor encompasses a processor that, in combination with additional processors, executes some or all code from one or more modules. The term shared memory encompasses a single memory that stores some or all code from multiple modules. The term group memory encompasses a memory that, in combination with additional memories, stores some or all code from one or more modules. The term memory may be a subset of the term computer-readable medium. The term computer-readable medium does not encompass transitory electrical and electromagnetic signals propagating through a medium and may, therefore, be considered tangible and non-transitory. Non-limiting examples of a non-transitory tangible computer readable medium include nonvolatile memory, volatile memory, magnetic storage, and optical storage.

The apparatuses and methods described in this application may be partially or fully implemented by one or more computer programs executed by one or more processors. The computer programs include processor-executable instructions that are stored on at least one non-transitory tangible computer readable medium. The computer programs may also include and/or rely on stored data.

What is claimed is:

1. An apparatus for disinfecting forced air systems, comprising:
a main member including an inner surface defining an aperture configured to receiving the forced air, the main member including a plurality of flanges disposed along the inner surface;
a UV light source to emit UV radiation into the aperture;
a sensor configured to detect movement of air through the aperture and provide a first signal thereof;
at least one light emission controller coupled to the sensor and turn on the light when the first signal is detected;
a filter member coupled to the flanges.

2. The apparatus as recited in claim 1, further comprising a TiO₂ configured to be illuminated by the UV radiation.

3. The apparatus as recited in claim 1, wherein the main member comprises an insulative layer.

4. The apparatus as recited in claim 1, wherein the main member comprises an acrylic light pile disposed between the UV light source and the aperture.

5. The apparatus as recited in claim 4, wherein the main member comprises a plurality of floor engaging support flanges.

6. The apparatus as recited in claim 1, wherein the apertures has a first portion defining an internal surface and wherein the filter has an exterior surface which is a complementary to the first surface.

7. The apparatus as recited in claim 1, wherein the air source includes a furnace for heating air emitted from the air source.

8. The apparatus as recited in claim 1, wherein the main member comprises a UV light source transmitting light through at least one optical light pipe.

9. The apparatus as recited in claim 8, wherein at least one aperture of the main member receives an end of the light pipe, the end emitting the UV light.

10. The apparatus as recited in claim 1, wherein the main member includes at least one horizontally oriented channel for creating an air-flow space between the main member and the forced air systems.

11. The apparatus as recited in claim 1, wherein the main member includes a plurality of racks for holding at least one filter.

12. The apparatus as recited in claim 11, wherein the flanges comprise a coupling mechanism which allows selective engagement of the filter to the interior surface.

13. An apparatus for disinfecting forced air systems, comprising:
an air deflector having an optically transparent to UV light polymer main member including an inner surface defining an inner surface configured for receiving a forced air stream from a forced air system;
a sensor disposed within the forced air stream, the sensor configured to produce a signal indicative of the presence of the air stream;
a UV light source optically coupled to the optically transparent to UV light polymer main member;
a controller configured to control the UV light source in response to the signal.

14. The apparatus as recited in claim 13, wherein the main member includes a first member for receiving a filter.

15. The apparatus as recited in claim 13, further comprising a disinfecting monochromatic light source.

16. The apparatus as recited in claim 13, further comprising an ozone source, and the ozone source is in communication with the main member.

17. A method of disinfecting particulates in a forced air stream in a forced air systems, comprising the steps of:
placing on a floor vent a main member, the main member including a plurality of flanges disposed along an inner surface, at least one light source for emitting a UV-C light, and at least one air source for blowing air;
disinfecting the particulates forced air systems using ozone from an ozone source; and
emitting UV-C light from the light source within the main member to illuminate the particulates.

18. The method as recited in claim 17, further comprising the step of illuminating the particulates with UV-C light.
